(19) United States
(12) Patent Application Publication
(10) Pub. No.: US 2006/0216318 A1
(43) Pub. Date: Sep. 28, 2006

(54) Compositions comprising entrained water and agents entrained in water-sensitive matrices

(76) Inventor: Gopa Majmudar, Irving, TX (US)

Correspondence Address: FULBRIGHT & JAWORSKI L.L.P., 600 CONGRESS AVE., SUITE 2400, AUSTIN, TX, 78701, US

(21) Appl. No.: 11/372,756

(22) Filed: Mar. 10, 2006

Related U.S. Application Data
(60) Provisional application No. 60/660,259, filed on Mar. 10, 2005.

(51) Int. Cl. A61K 8/02

(52) U.S. Cl. 424/401

(57) ABSTRACT

The present invention provides compositions comprising a water-sensitive matrix, a water-dispersible agent, and/or entrained water. The entrained water, for example, can be entrained within a plurality of capsules or particles, wherein upon use the entrained water is released thereby effecting the release of the water-dispersible agents. The invention also provides methods of use for the topical application of such compositions.

This application claims the benefit of U.S. Provisional Application Ser. No. 60/660,259, filed Mar. 10, 2005, the contents of which are incorporated by reference.

### BACKGROUND OF THE INVENTION

1. Field of the Invention

The present invention relates generally to methods of delivery of water dispersible agents, including cosmetic agents. In particular, the present invention is directed to compositions comprising a water-dispersible agent, a water-sensitive matrix, and entrained water. These compositions, for example, can be used to deliver water-dispersible agents to a person's skin.

2. Background

The cosmetic industry continually searches for ways to enhance the performance of consumer and cosmetic products and make them more aesthetically pleasing for the consumers. Consumer acceptance of cosmetic products is determined not only by the performance achieved with these products but also the aesthetics associated therewith. Water provides a number of purposes, both functional and aesthetic, in cosmetic and consumer product preparations. For example, it provides a suitable solvent for various ingredients, provides hydration of the skin, and can provide a skin cleaning function, e.g., with skin wipes. However, use of water may also engender numerous problems, such as the instability of various compounds in aqueous solution, e.g., vitamin C, and packaging problems, e.g., drying of wet skin wipes. There is currently a need for improvements in providing water in a controlled fashion to cosmetic and consumer product preparations.

### SUMMARY OF THE INVENTION

One aspect of the present invention is a topical composition comprising a water-sensitive matrix, which may be a water-soluble film, and a water-dispersible agent, which may be hydrophilic or hydrophobic, and which may be a cosmetic agent. In non-limiting aspects, the composition can also include entrained water. In non-limiting embodiments, the water can be entrained in, for example, a capsule or particle.

Another aspect of the present invention is a cosmetic or skin care product comprising a dehydrated or encapsulated cosmetic or skin care active component dispersed within a matrix of a liquid soluble film. In some embodiments, the product is combined with sufficient liquid to dissolve the film and release the cosmetic or skin care active components. In some embodiments, the liquid is water. The invention includes liquid soluble films comprising cosmetic or skin care active agents that are provided in conjunction with entrained liquid that can dissolve the films during use and also such films that are provided without such entrained liquid.

A further aspect of the present invention is a composition comprising a water-dispersible agent entrained within a water-sensitive matrix and water entrained within a plurality of capsules or particles. For the purposes of the present invention, “water-sensitive matrix” means a matrix that upon contact with water will dissolve or partially dissolve or otherwise physically alter such as to allow for the dispersion of a substantial amount of a water-dispersible agent. “Water-dispersible agent” means an agent dispersed by the action of water on the water-sensitive matrix, regardless of whether such agent is soluble, partly soluble, or substantially insoluble in the aqueous milieu that causes such dispersion. As such, the term “water-dispersible agents” encompasses agents that may be hydrophobic or hydrophilic in nature. In some embodiments, the composition is adapted for topical use. Topical use includes application to the skin via conventional vehicles such as creams, lotions gels, etc., and also the application to skin from agents released from films, e.g., wherein such agents are released by subjecting a film to a physical force (nonlimiting examples include crumpling, squeezing, rubbing, pressing, tearing, etc.) wherein the released water-dispersible agent is then either directly applied to the skin by using the film as a wipe, or indirectly applied to the skin by transfer to a hand and then rubbed onto the skin. The amount of water entrained within the plurality of capsules or particles is sufficient to dissolve or otherwise effect the water-sensitive matrix such as to result in the release of a substantial amount of a water dispersible agent. In some embodiments, the water-dispersible agent is a cosmetic agent, which may be a skin care active agent.

In some embodiments, the water sensitive matrix comprises one or more water soluble polymer, which may be in the form of, or be a component of, a water soluble film or water soluble microspheres. In some embodiments, the water is entrained in a plurality of silica particles or is entrained in a plurality of breakable capsules. In some embodiments, the plurality of capsules or particles with water entrained within them or with water-dispersible agents entrained within them, or both, are adapted to adhere to at least one surface of a water soluble film. In some embodiments, the plurality of capsules or particles comprise a middle layer, or component of a middle layer, positioned between a first water soluble film layer and a second layer. In some embodiments, the second layer is substantially impermeable to water and in some embodiments the second layer is a second water soluble film layer.

In some embodiments, wherein the one or more water soluble polymer is in the form of a water soluble microsphere, or a component thereof, the composition comprises a component of an otherwise anhydrous cream, gel, lotion, or ointment.

In some embodiments, the water-dispersible agent is a cosmetic agent. In further embodiments, the plurality of capsules or particles with entrained water further comprise a cosmetic agent.

One aspect of the present invention is a topical composition comprising a water-sensitive film and a water-dispersible cosmetic agent. In some embodiments, the composition further comprises water entrained within a plurality of capsules or particles.

In certain non limiting embodiments, the entrained water can be entrained within, for example, a fumed silica particle, a capsule, or a particle. The capsule or particle can be breakable or dissolvable. The fumed silica particle, capsule, or particle can, be for example, dispersed in the water-sensitive matrix and/or adhered to at least one surface of the water-sensitive matrix. In certain aspects, the fumed silica particle, capsule, or particle can be adhered to a second surface of the water-sensitive matrix. The fumed silica particle, capsule, or particle, in certain aspects, comprises a middle layer that is positioned between the water-sensitive matrix and a second layer. The second layer can include a water-sensitive matrix.

In still other aspects, the water-dispersible agent can be entrained within a capsule or particle or dispersed in the water-sensitive matrix. Similarly, the entrained water and the water-dispersible agent can be entrained within the same capsule or particle. The capsule or particle can be breakable or dissolvable. The capsule or particle can be adhered to at least one or two surfaces of the water-sensitive matrix, for example. In certain embodiments, the capsule or particle can dispersed in the water-sensitive matrix.

In other aspects, the water-sensitive matrix can include a dye. The dye can include a color. Non-limiting examples of colors include pink, blue, green, red, yellow, orange, black, grey, or purple. The water-sensitive matrix can include a writing or graphics on its surface.

Another aspect of the present invention is a process for the topical application of a water-dispersible agent, comprising providing a subject with a composition comprising a water-dispersible agent entrained within a water-sensitive matrix and water entrained within a plurality of capsules or particles. The subject applies physical force to the composition sufficient to release the water entrained in the plurality of capsules or particles, and the released water dissolves or otherwise effects the water-sensitive matrix such as to release at least a substantial amount of the water dispersible agent.

In some embodiments, the water sensitive matrix comprises one or more water soluble polymers, which in some embodiments may be in the form of a water soluble film or water soluble microsphere, or components thereof. In some embodiments, the water is entrained within a plurality of silica particles or within a plurality of breakable capsules. In some embodiments, the plurality of capsules or particles with entrained water are adapted to adhere to at least one surface of a water soluble film. In further embodiments, the plurality of capsules or particles comprise a middle layer, or component thereof, positioned between a first water soluble film layer and a second layer. In some embodiments, the second layer is substantially impermeable to water and in some embodiments the second layer is a second water soluble film layer. In some simple embodiments, the entrained water and the cosmetic agent are both comprised within the water-soluble matrix.

In some embodiments wherein the one or more water soluble polymer is in the form of a water soluble microsphere, or a component thereof, the composition comprises a component of an otherwise anhydrous cream, gel, lotion, or ointment.

In some embodiments the water-dispersible agent is a cosmetic agent. In further embodiments, the plurality of capsules or particles with entrained water further comprise a cosmetic agent.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

A more complete understanding of the method and apparatus of the present invention may be obtained by reference to the following detailed description of the invention, with like reference numerals denoting like elements, when taken in conjunction with the accompanying drawings wherein:

FIG. 1 depicts a side plan view of a composition of the present invention wherein capsules or particles with water entrained within them are adapted to adhere to the surface a water soluble film 100.

FIG. 2 depicts a side plan view of a trilaminate composition of the present invention.

FIG. 3 depicts a side plan view of a bilaminate composition of the present invention.

FIG. 4 depicts a side view of a single layer composition of the present invention.

### DETAILED DESCRIPTION OF ILLUSTRATIVE EMBODIMENTS

In certain aspects, the present invention provides compositions including a water-dispersible agent, entrained water, and/or a water-sensitive matrix, wherein upon during use the entrained water is released, thereby effecting the release of the water-dispersible agents. The invention also provides methods of use for topical application of such compositions. These and other aspects of the present invention are described in further detail below.

A. Water-Sensitive Matrices

A number of synthetic and natural polymers known in the art provide suitable water-sensitive matrices for the present invention.

1. Water Soluble Films

Examples of dissolvable polymer films are provided by U.S. Patent Application Publications No. 20020161088, 20040071755, 20050019588, all incorporated herein by reference. Suitable film forming polymers include polymers selected from pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, pectin, tragacanth gum, guar gum, acacia gum, Arabic gum, polyacrylic acid, methylmethacrylate copolymer, carboxyvinyl polymer, gelatin, amylase, high amylase starch, hydroxypropylated high amylase starch, dextrin, chitin, chitosan, levan, elsinan, collagen, zein, gluten, soy protein isolate, whey protein isolate, casein and mixtures thereof.

To improve flexibility and reduce brittleness of the film compositions, a softener or plasticizing agent may be used to improve the flexibility of films, the amount of which may vary up to about 20% dry weight of the film composition. The amount of plasticizer used should be excessive in that the film loses structural integrity or becomes sticky such that it tends to adhere to other strips of film. Suitable plasticizing agents include, but are not limited to, polyols such as sorbitol, glycerin, polyethylene glycol, propylene glycol, monoacetin, diacetin, triacetin, hydrogenated starch hydrolysates, corn syrups, derivatives thereof and combinations thereof. A polyalcohol may be used to achieve the desired level of softness of the film. Examples of polyalcohols include glycerol, polyethylene glycol, propylene glycol, glycerol monoesters with fatty acids or other pharmaceutically used polyalcohols. The concentration of the polyalcohol in the dry film may range between 0.1 and 5% (w/w). Some plasticizers are water soluble and are miscible with a polymer.

Surfactants may optionally be included in the films. Suitable surfactants include water-soluble anionic surfactants such as carboxylate soaps, alkylarylsulfonates, alkanesulfonates, alpha-olefinsulfonates, fatty alcohol sulfates, and oxo-alcohol ether sulfates. Suitable nonionic surfactants include the alkylphenol ethoxylates, fatty or oxo-alcohol polyethylene glycol ethers, ethylene oxide-propylene oxide block copolymers, fatty alcohol polygylcol ethers and ethoxylated fatty alcohols. The surfactants may be used in amounts normally effective to assist in dispersion of water-soluble polymers, such as about 1% to about 30% by weight of total film-forming composition.

Optional antifoam agents include the silicon polymers, and defoamers include tallow compounds. Optional stabilizing agents include xanthan gum, locust bean gum and carrageenan, in amounts ranging from about 0 to about 10 wt % or otherwise. Other suitable stabilizing agents include guar gum and the like. Optional emulsifiers may be included in the film, e.g., casein, triethanolamine stearate, quaternary ammonium compounds, acacia, gelatin, lecithin, bentonite, veegum, and the like. Amounts typically range from about 0 to about 5 wt % or otherwise.

The film-forming compositions can be made into films of varying thickness on a commercial scale using conventional methods and techniques such as solution casting and thermo-forming techniques. Films prepared from the film-forming compositions of the present invention may be in the range of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 160, 170, 180, 190, or 200 microns or greater in thickness. In solution casting, the compositions are cast on a plate or belt using a film applicator where they are allowed to dry. The films can then be vacuum-dried, followed by removal from the coating plate/belt. Casting techniques are described in U.S. Pat. No. 5,272,191, incorporated herein by reference. Melt processes for forming films typically involve mixing the components together with sufficient water to allow the polymer and principal solvent to melt at a temperature below their decomposition temperatures. The blended material is fed to an extruder, extruded under tension through an appropriate die, cooled with air and taken up by an appropriate collection device. Extrusion processes can be used to make desired shaped articles by using appropriate dies and molds. Examples of such thermo-forming processes are described in U.S. Pat. No. 5,646,206, incorporated herein by reference.

Water-dispersible agents of the present invention, such as cosmetic agents, may be included as an additional component in the water-sensitive film.

2. Water Sensitive Microspheres

Examples of water sensitive microspheres suitable for encapsulating water-dispersible agents of the present invention, are provided in U.S. Patent Publications No. 20030194416 and 20030198652, both incorporated herein by reference. Suitable water-sensitive materials for forming the microspheres include polyvinyl pyrrolidone, water soluble celluloses, ethylene maleic anhydride copolymer, methylvinyl ether maleic anhydride copolymer, acrylic acid copolymers, anionic polymers of methacrylic acid and methacrylate, cationic polymers with dimethyl-aminoethyl ammonium functional groups, polyethylene oxides, water soluble polyamide or polyester.

Examples of water soluble hydroxyalkyl and carboxyalkyl celluloses include hydroxyethyl and carboxymethyl cellulose, hydroxyethyl and carboxyethyl cellulose, hydroxymethyl and carboxymethyl cellulose, hydroxypropyl carboxymethyl cellulose, hydroxypropyl methyl carboxyethyl cellulose, hydroxypropyl carboxypropyl cellulose, hydroxybutyl carboxymethyl cellulose, and the like. Also useful are alkali metal salts of these carboxyalkyl celluloses, particularly and preferably the sodium and potassium derivatives.

A suitable polyvinyl alcohol is partially and fully hydrolyzed polyvinyl acetate, termed “polyvinyl alcohol” with polyvinyl acetate hydrolyzed to an extent of from about 75% up to about 99%. The preparations of such materials are exemplified in U.S. Pat. No. 5,051,222, incorporated herein by reference.

Suitable polysaccharides are polysaccharides of the non-sweet, coloidally-soluble types, such as natural gums, for example, gum arabic, starch derivates, dextrinized and hydrolyzed starches, and the like. A suitable polysaccharide is a water dispersible, modified starch commercially available as Capul®, N-Lok®, Hi-Cap™ 100 or Hi-Cap™ 200 commercially available from the National Starch and Chemical Company of Bridgewater, N.J.; Pure-Cote™, commercially available from the Grain-Processing Corporation of Muscatine, Iowa. In the preferred embodiment, the natural gum is a gum arabic, commercially available from TIC Gums Inc. Belcamp, Midland. Suitable hydrocolloids are xanthan, maltodextrin, galactomanan or tragacanth, preferably maltodextrins such as Maltrin™ 100, and Maltrin™ M150, commercially available from the Grain Processing Corporation of Muscatine, Iowa.

One method by which the microspheres of the present invention can be prepared is by the following steps: forming an aqueous phase of the moisture sensitive materials (either a single material or mixture of several materials); emulsifying the water-dispersible agent or agents in the aqueous phase; and removing moisture to create free-flowing powder. For example, moisture can be removed by spray drying droplets of emulsion. Spray drying is well known in the art and has been used commercially in many applications, including foods where the core material is a flavoring oil and cosmetics where the core material is a fragrance oil, as described in Cf. Balassa, “Microencapsulation in the Food Industry”, CRC Critical Review Journal in Food Technology, July 1971, pp 245-265; Barreto, “Spray Dried Perfumes for Specialties, Soap and Chemical Specialties”, December 1966; Maleeny, Spray Dried Perfumes, Soap and San Chem, January 1958, pp. 135 et seq.; Flinn and Nack, “Advances in Microencapsulation Techniques”, Batelle Technical Review, Vo. 16, No. 2, pp. 2-8 (1967); U.S. Pat. Nos. 5,525,367; and 5,417,153 which are incorporated herein as references.

B. Entrained Water

One non-limiting suitable form of entrained water is Aqua Foundation, commercially available from Degussa AG. This product is based on surface modified fumed silica, e.g., AEROSIL® R 812 (silica silylate) (Hasenzhal & Braungel, SÖFW-Journal, “Fumed silica for personal care and cosmetics—versatile and effective,” 129:2-8, 2003). Water droplets introduced into the presence of hydrophobic fumed silica during a mixing process are coated by the silica, resulting in a powdered product, so-called “dry water.” Formulations can contain up to 80% by weight. Under mechanical stress, such as when the product is rubbed on skin, the water is released.

Other suitable forms of entrained water include breakable capsules. Non-limiting examples of breakable capsules are provided in U.S. Pat. No. 6,508,604, incorporated herein by reference and by U.S. Application Publication No. 20030084914, incorporated herein by reference. Water may be released from the breakable capsules of the present invention by rubbing topical compositions on the skin or by the crumpling of film compositions to which breakable capsules have been incorporated.

In certain non-limiting embodiments, the present compositions may comprise in their final form, for example, at least about 1%, 5%, 10%, 15%, 20%, 25%, 30%, 35%, 40%, 45%, 50%, 55%, 60%, 65%, 70%, 75%, or 80% or more by weight of water, and any range derivable therein.

C. Water-Dispersible Agents

Water-dispersible agents include cosmetic agents, which may be hydrophobic agents, such as waxes, oils, petrolatum, silicon products and fatty acids, or may be hydrophilic agents. In certain non-limiting aspects, the compositions of the present invention can be applied to the skin without undue toxicity, incompatibility, instability, allergic response, and the like. The term “cosmetic agent” also includes “skin care active” agents, i.e., agents directed to enhancing, modifying or maintaining a biological or physiological functionality or as being used for a purely aesthetic effect.

In regard to cosmetic agents generally, the CTFA Cosmetic Ingredient Handbook, Second Edition (1992) describes a wide variety of non-limiting cosmetic ingredients commonly used in the skin care industry, generally suitable for use in the compositions of the present invention. Examples of these ingredient classes include: aesthetic components such as fragrances, pigments, colorings/colorants, essential oils, skin sensates, anti-acne agents, anti-caking agents, antimicrobial agents, antioxidants, chelating agents, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives, aloe vera, pantothenic acid and its derivatives, allantoin, bisabolol, and dipotassium glycyrrhizinate), skin treating agents, thickeners, and vitamins and derivatives thereof.

3. Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

As is evident to those of skill in the art, moisturizing agents may be provided in suitable formulations. A non-limiting example of such a formulations includes a mixture of shea butter (20% by weight), mineral oil (40% by weight) and Soya soft Skin 567 (40% by weight, available from DPS Wax Inc.). The ingredients are added together, heated to 75° C. while mixing, held at temperature for 20 minutes and then cooled to room temperature. Another suitable formulation includes: water (26.79% by weight), glycerine (35.71% by weight), sodium PCA (0.89% by weight), Prodew 400 (0.89% by weight, available from Ajinamoto) and Betafin BP-20 (35.71% by weight, available from Arch Personal Products).

4. Antioxidants

Non-limiting examples of antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

5. Ultraviolet Light Absorbing Compounds

Non-limiting examples of compounds that have ultraviolet light absorbing properties that can be used with the compounds of the present invention include titanium dioxide, zinc oxide, benzophenone, benzophenone-1, benzophenone-2, benzophenone-3, benzophenone-4 benzophenone-5, benzophenone-6, benzophenone-7, benzophenone-8, benzophenone-9, benzophenone-10, benzophenone-11, benzophenone-12, benzyl salicylate, butyl PABA, cinnamate esters, cinoxate, DEA-methoxycinnamate, diisopropyl methyl cinnamate, ethyl dihydroxypropyl PABA, ethyl diisopropylcinnamate, ethyl methoxycinnamate, ethyl PABA, ethyl urocanate, glyceryl octanoate dimethoxycinnamate, glyceryl PABA, glycol salicylate, homosalate, isoamyl p-methoxycinnamate, PABA, PABA esters, Parsol 1789, and isopropylbenzyl salicylate.

6. Additional Water-Dispersible Agents

Non-limiting examples of additional compounds and agents that can be used with the compositions of the present invention include skin lightening agents (e.g. kojic acid, hydroquinone, ascorbic acid and derivatives, retinoids and their derivatives, and niacinamide), emollients (e.g., esters and fatty acids), vitamins (e.g., D, E, A, K, and C), trace metals (e.g., zinc, calcium and selenium), anti-irritants (e.g., steroids and non-steroidal anti-inflammatories), antimicrobial agents (e.g., triclosan), botanical extracts (e.g., aloe vera, chamomile, cucumber extract, ginkgo bibloba, ginseng, and rosemary), dyes and color ingredients (e.g., D&C blue no. 4, D&C green no. 5, D&C orange no. 4, D&C red no. 17, D&C red no. 33, D&C violet no. 2, D&C yellow no. 10, D&C yellow no. 11 and DEA-cetyl phosphate), preservatives (e.g., BHA), emollients (i.e., organic esters, fatty acids, lanolin and its derivatives, plant and animal oils and fats, and di- and triglycerides), antimicrobial agents (e.g., triclosan and ethanol), and fragrances (natural and artificial).

D. Cosmetic Products

A person of ordinary skill would recognize that the compositions of the present invention can include any number of combinations of water-dispersible agents. It is also contemplated that the concentrations of the water-dispersible agents of the present invention can vary. In certain non-limiting embodiments, the present compositions may comprise in their final form, for example, by at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 30%, 35%, 40%, 50%, or 60%, or more by weight of at least one water-dispersible agent, and any range derivable therein.

Non-limiting examples of suitable cosmetic vehicles that may contain water-dispersible agents entrained in water soluble microspheres include otherwise anhydrous suitable cosmetic creams, gels, lotions, and ointments, as are known in the art, or by other suitable methods or any combination of the foregoing as would be known to one of ordinary skill in the art (Remington's, Pharmaceutical Sciences, 18th Ed. Mack Printing Company, 1990). Variations and other appropriate vehicles will be apparent to the skilled artisan and are appropriate for use in the present invention. Non-limiting examples of suitable film compositions for use are disclosed in Section A. 1 of the present disclosure.

A person of ordinary skill would recognize that the compositions of the present invention can be incorporated into many different product types. Non-limiting examples include: demonstration or sampler products; fragrance patches; soaps; masks and/or cleansers, including oil control agents and de-stress ingredients such as aromatherapy; and a lip primer for the treatment of fine lines on lips, incorporating antiaging ingredients as known in the art. Other treatment product types include, but are not limited to: moisturization booster for the eye and/or face; hand cream; acne treatments, including agents such as benzoylperoxide, azelaic acid, adapalene, tretinoin, clindamycin, erythromycin, and erythromycin-zinc complex, and agents that alleviate acne-associated redness and irritation; skin lightening and skin whitening agents; antiaging agents for the treatment of fine lines and wrinkles; and cover-up products for skin imperfections such as dark spots, wrinkles and pimples.

In the foregoing products, it is important that the concentrations and combinations of the compounds and ingredients be selected in such a way that the combinations are chemically compatible and do not form precipitates or otherwise cause instability of the finished product.

E. Configuration of Products

There are a wide variety of configurations of combinations of water-dispersable agents in water soluble matrices in conjunction with entrained water. Some of these are shown in the figures and discussed below. However, those of skill will recognize that a wide variety of configurations are possible.

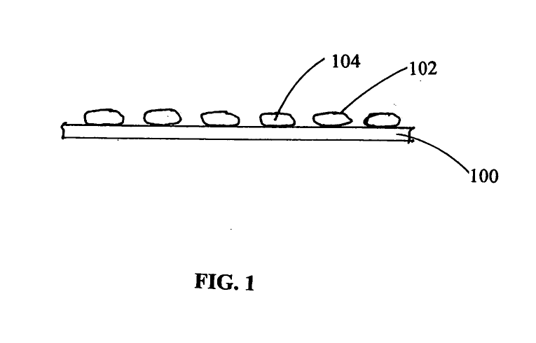

In some embodiments of the present invention, as shown in FIG. 1, the capsules or particles 102 with water 104 entrained within them are adapted to adhere to at least one surface of a water soluble film 100.

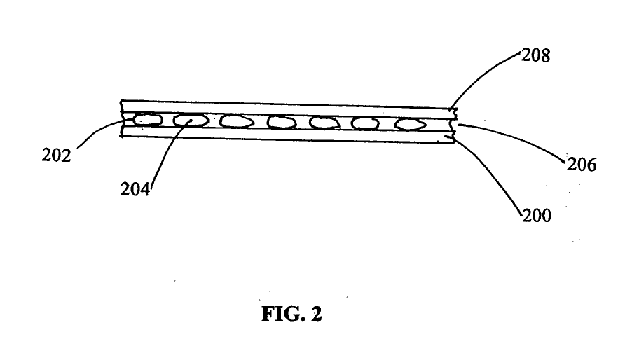

In other embodiments, the water soluble films are a component of a laminate composition, e.g., a trilaminate as shown in FIG. 2, wherein the capsules or particles 202 with water 204 entrained within them comprise a middle layer 206, or component thereof, that is positioned between a first water soluble film layer 200 and a second layer 208. In some embodiments the second layer 208 is substantially impermeable to water while in some embodiments the second layer 208 is a second water soluble film layer.

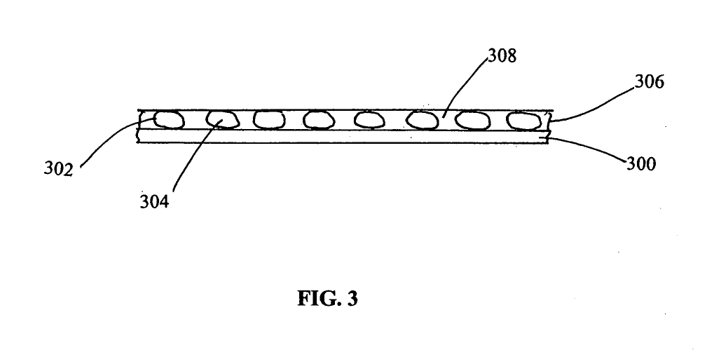

FIG. 3 depicts an example of a bilaminate structure, wherein the capsules or particles 302 with water 304 entrained within are a component of a first layer 306 and surrounded by a matrix 308, and is adjacent a second water soluble film layer 300. Laminate composition of this nature can be prepared by methods known generally in the art.

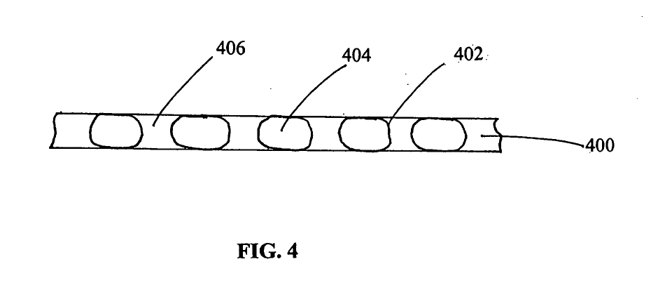

FIG. 4 depicts a structure, wherein the capsules or particles 402 with water 404 entrained within are a component of a film 400 and where they are surrounded by a matrix 406.

F. Effect of Water on Water-Sensitive Matrices

The effect of water on water-sensitive matrices may suitably be estimated by titrating increasing amounts of water, preferably at room temperature, on a standard amount of either water-soluble film or water soluble microspheres. The effect may be estimated visually or the dissolution of a water dispersible tracer agent may be estimated. The latter may be suitably performed by placing the water soluble film or microspheres containing a water dispersable tracer agent on filter paper, titrating with water, and at set times remove the filter paper and extract and quantify the tracer agent by suitable analytical methods known in the art. The results indicate both the rapidity of dispersal of agents entrained within the water-sensitive matrix and also indicate the suitable amount of entrained water to be incorporated into the product.

The above text describes preferred embodiments of the invention. It should be appreciated by those of skill in the art that many changes can be made in the specific embodiments which are disclosed, and still obtain a like or similar result without departing from the spirit and scope of the invention.

1. A composition comprising:
(a) a water-sensitive matrix;
(b) a water-dispersible agent; and
(c) entrained water.

2. The composition of claim 1, wherein said composition is adapted for topical application.

3. The composition of claim 1, wherein said water-sensitive matrix comprises one or more water soluble polymers.

4. The composition of claim 3, wherein said one or more water soluble polymers is in the form of a water soluble film, or a component thereof, or is in the form of water soluble microspheres, or a component thereof.

5. The composition of claim 4, wherein said one or more water soluble polymer is in the form of water soluble microspheres, or a component thereof.

6. The composition of claim 5, wherein said composition comprises a component of an otherwise anhydrous cream, gel, lotion, or ointment.

7. The composition of claim 1, wherein said entrained water is entrained within a fumed silica particle, a capsule, or a particle.

8. The composition of claim 7, wherein said capsule or particle is breakable or dissolvable.

9. The composition of claim 7, wherein the fumed silica particle, capsule, or particle is dispersed in the water-sensitive matrix.

10. The composition of claim 7, wherein the fumed silica particle, capsule, or particle is adhered to at least one surface of the water-sensitive matrix.

11. The composition of claim 10, wherein the fumed silica particle, capsule, or particle is adhered to a second surface of the water-sensitive matrix.

12. The composition of claim 7, wherein the fumed silica particle, capsule, or particle comprise a middle layer that is positioned between the water-sensitive matrix and a second layer.

13. The composition of claim 12, wherein said second layer comprises a water-sensitive matrix.

14. The composition of claim 1, wherein said water-dispersible agent is a cosmetic agent.

15. The composition of claim 1, wherein the water-dispersible agent is entrained within a capsule or particle.

16. The composition of claim 15, wherein the capsule or particle is breakable or dissolvable.

17. The composition of claim 15, wherein the capsule or particle is adhered to at least one surface of the water-sensitive matrix.

18. The composition of claim 17, wherein the capsule or particle is adhered to a second surface of the water-sensitive matrix.

19. The composition of claim 15, wherein the capsule or particle is dispersed in the water-sensitive matrix.

20. The composition of claim 1, wherein the entrained water and the water-dispersible agent is entrained within the same capsule or particle.

21. The composition of claim 20, wherein the capsule or particle is breakable or dissolvable.

22. The composition of claim 20, wherein the capsule or particle is adhered to at least one surface of the water-sensitive matrix.

23. The composition of claim 22, wherein the capsule or particle is adhered to a second surface of the water-sensitive matrix.

24. The composition of claim 20, wherein the capsule or particle is dispersed in the water-sensitive matrix.

25. The composition of claim 1, wherein the water-sensitive matrix comprises a dye.

26. The composition of claim 25, wherein the dye is pink.

27. The composition of claim 1, wherein the water-sensitive matrix comprises writing or graphics on a surface of the water-sensitive matrix.

28-30. (canceled)

31. A process for topical application of a water-dispersible agent comprising providing a subject with a composition comprising:
(a) a water-sensitive matrix;
(b) a water-dispersible agent; and
(c) entrained water,
wherein the subject applies physical force to the composition sufficient to release said entrained water, and said released water dissolves or otherwise effects the water-sensitive matrix such as to release a substantial amount of said water dispersible agent.

32-44. (canceled)
